(19) United States
(12) Patent Application Publication
(10) Pub. No.: US 2004/0068228 A1
(43) Pub. Date: Apr. 8, 2004

(54) Device and method for stabilizing catheters

(76) Inventor: Jon Cunningham, Cumming, GA (US)

Correspondence Address: TECHNOPROP COLTON, L.L.C., P O BOX 567685, ATLANTA, GA, 311567685

(21) Appl. No.: 10/264,567

(22) Filed: Oct. 4, 2002

(51) Int. Cl. A61M 29/00

(52) U.S. Cl. 604/104; 604/175

(57) ABSTRACT

A stabilizing device that can be configured with a catheter such that the stabilizing device can apply pressure to the surrounding tissue, which thereby can help stabilize the catheter in place, and methods for employing the stabilizing device to stabilize catheters within a patient.

### BACKGROUND OF THE INVENTION

1. Technical Field

The present invention relates generally to the field of stabilizing devices useful for stabilizing catheters within a body. The present invention generally is for use in combination with catheters for use in applications that require blood, fluids, medicated solutions and other solutions to be removed from and/or introduced into a person. The present invention relates more particularly to the field of stabilizing devices for hemodialysis catheters, the stabilizing devices having an expandable or inflatable stabilizing chamber that is positioned within the subcutaneous tunnel created through a patient's skin and tissues for the insertion of the catheter into the patient's body. The present invention also relates to methods for constructing such devices and for utilizing such devices to stabilize hemodialysis catheters.

2. Prior Art

Hemodialysis is a procedure that generally requires the introduction and removal of blood from a patient, and is a routine treatment for patients with renal failure. When patients are placed on hemodialysis, such patients often require the placement of a catheter into a large vein at the base of the neck through a subcutaneous tunnel. In conventional procedures, including the well-known Seldinger technique, the catheter is inserted through the patient's skin and into a blood vessel. This catheter, termed a hemodialysis catheter, is connected to a hemodialysis machine and is the vital connection between the patient and the hemodialysis machine. Once the practitioner properly inserts the hemodialysis catheter, reliable hemodialysis can be performed for weeks to months using the placed catheter. During this time while the catheter is in the patient, it is necessary to stabilize the hemodialysis catheter relative to the patient's subcutaenous tissue and the subcutaneous tunnel to prevent its movement and to maintain its position.

The prior art discloses a myraid of means for stabilizing a catheter in place relative to the patient's subcutaneous tissue or skin. An example of one such means is through the use sutures, namely, the catheter assembly is sutured directly to the epidermis tissue. Another example of one such means is through the use of tapes, namely, an external portion of the catheter assembly is taped to the patient's skin. Yet another example of one such means is through the use of a fabric cuff that is located about the catheter and in which the subcutaneous tissue grows.

On the one hand, a destabilized or unstable catheter can become loose and can create potential complications to the patient such as infection or irritation at the point of catheter insertion. Specifically, a destabilized catheter can move relative to the patient, sliding within, or moving laterally against the sides of, the subcutaneous tunnel. In more extreme cases, an unsupported catheter can create exit site (where the catheter exits the patient's body) complications that can require the catheter to be replaced and/or extensive surgery to correct. Thus, it is necessary for a catheter to be relatively stable within the patient.

On the other hand, a catheter that is substantially attached or excessively anchored to the skin or to the subcutaneous tissue within the subcutaneous tunnel also can create complications. For example, if the catheter is tightly positioned and has an excessively large stabilizing device within the subcutaneous tissue, the practitioner may have problems inserting and removing the catheter from the patient. In some cases, as conventional catheters are generally composed of material having a relatively low tensile strength, an excessively anchored catheter can break or tear during such procedures, thus requiring additional techniques and/or surgery to correct.

Accordingly, there is always a need for improved devices and methods for stablizing a catheter, such as a hemodialysis catheter, in place relative to a patient's skin or subcutaneous tissue within a subcutaneous tunnel. Further, there also is a need for improved devices and methods for stabilizing a catheter that allow for easier removal and insertion of the catheter out of and into the patient's body. It is to these and other needs that the present invention is directed.

### BRIEF SUMMARY OF THE INVENTION

Briefly, the present invention includes a stabilizing device that can be configured with a catheter, such that the stabilizing device can cooperate with the surrounding subcutaneous tissue within the subcutaneous tunnel, and thereby can help stabilize the catheter in place within the subcutaneous tunnel. The present invention also includes catheters comprising the stabilizing device and methods for employing the stabilizing device to stabilize catheters.

The present invention is a stabilizing device that can be configured and used in conjunction with any type of catheter that is inserted through the skin of a patient, such as those used in hemodialysis procedures. Summarily, the stabilizing device of the present invention comprises an expandable or inflatable chamber having an exterior surrounding surface and a means for inflating the chamber. In one embodiment, the chamber is dimensioned to surround circumferentially at least a portion of the catheter. Generally, the means for inflating the chamber is positioned external to the exit site with respect to the patient as such means is employed by the practitioner to control the expansion or inflation of the chamber. The chamber generally is positioned on the catheter such that when the catheter is positioned in the subcutaneous tunnel, the chamber is positioned external to the catheterized part of the blood vessel, but internal to the skin exit site, that is, within the patient's body between the skin surface and the blood vessel. When the chamber is expanded or inflated, it can apply pressure to the subcutaneous tissue and thereby help stabilize the catheter within the subcutaneous tissue.

Preferably, the placement of the stabilizing device on the catheter does not affect the function of the catheter. For example, the chamber preferably can be configured so that it does not occlude, obstruct, or interfere with the structure of the fluid lumens(s) of the catheter or the insertion or removal of the catheter into or out of the patient. Preferably, the chamber does not change the diameter of the fluid lumens(s) of the catheter in either the contracted (deflated) or expanded (inflated) states. More specifically, because the chamber preferably is positioned on the outside of the walls of the catheter external to the fluid lumens(s), the stabilizing device does not protrude into the catheter itself or interfere with the fluid flow within the catheter itself.

One means for expanding or inflating the chamber is through the use of an inflation port and a syringe or other type of injection device in order to expand said chamber. In this embodiment, the syringe is employed to introduce a biocompatible filler into the chamber. The syringe is filled with a biocompatible filler and is inserted into the inflation port, and the biocompatible filler is ejected from within the syringe into the chamber. Preferably, the inflation port has a valve that is closed when the syringe is not inserted therein and that is opened when the syringe is inserted therein. The biocompatible filler travels from the hypodermic needle through the valve, if present, and into the chamber, causing the chamber to expand or inflate. As the chamber expands or inflates, the exterior surrounding surface of the chamber, which may or may not already be in contact with the subcutaneous tissue within the subcutaneous tunnel, is forced against the subcutaneous tissue within the subcutaneous tunnel, thus stabilizing and/or anchoring the catheter within the subcutaneous tunnel.

A catheter comprising the stabilizing device can be easier to withdraw from the subcutaneous tunnel. After the chamber is contracted (deflated), the catheter can be removed from the patient without needless surgery. More specifically, after the subcutaneous tissue has been separated from the catheter and the configured stabilizing device, the practitioner can without undue impedance remove the catheter from the patient and thereafter close the area. One method of removing the catheter from the patient can be simply using the hand to withdraw the catheter from the subcutaneous tunnel. Because the subcutaneous tissue preferably does not grow into the exterior surrounding surface of the chamber in certain embodiments of the stabilizing device, it can be possible to slide the catheter out of the subcutaneous tunnel without any surgery, after which the practitioner can close the area.

In alternative embodiments of the invention, there can be optional end pieces of a tapered shape flanking the chamber, either on the insertion side or the removal side or both of the stabilizing device. The end pieces can be useful in promoting the passage of the chamber through the subcutaneous tunnel of the patient. More particularly, the tapered ends can help prevent the edges of the chamber from snagging, dragging or catching on the subcutaneous tissue. Preferably, the end pieces taper from being approximately flush with the catheter surface to being approximately flush with chamber when the chamber is in a contracted (deflated) state.

Further, the stabilizing device can be constructed of or include materials to prevent bacterial growth. For example, a portion of the stabilizing device can be coated with a Decron® polymer or other similar material to impede bacterial growth. Further, other materials such as silver or antimicrobial substances can be placed proximally to the chamber to prevent infection in the area of placement.

In operation and use, the stabilizing device is adapted to allow a practitioner to stabilize a catheter relative to a patient. Preferably, after the catheter has been inserted through the patient's skin into the appropriate blood vessel (by techniques such as the Seldinger technique) and the chamber has been appropriately placed within the subcutaneous tunnel for optimal positioning, the practitioner begins to expand (inflate) the chamber by injecting a biocompatible filler into the chamber. As the chamber expands (inflates), generally radially, proportional to the amount of biocompatible filler injected therein, the chamber eventually begins to apply pressure to the subcutaneous tissue. This pressure helps secure the chamber, and hence the catheter, to the subcutaneous tissue, and hence within the subcutaneous tunnel. When the practitioner needs to remove the catheter from the patient, the practitioner can contract (deflate) the chamber by withdrawing the biocompatible filler, which reduces the pressure on the subcutaneous tissue, and the catheter can removed.

Methods for manufacturing the stabilizing device are evident to those having ordinary skill in the art. In one embodiment, the catheter can be manufactured independently of the stabilizing device and the stabilizing device subsequently affixed to the catheter. For example, the stabilizing device can be manufactured using an ordinary molding method and later affixed to the catheter by means of an adhesive, ultrasonic welding, or other type of adhesive method obvious to those with skill in the art. In other embodiments, specific types of catheters can be manufactured with the stabilizing device so as to produce a single unit.

These features and advantages of the present invention and the complementary method for installing the invention will become more apparent to those of ordinary skill in the art when the following detailed description of the preferred embodiments is read in conjunction with the appended figures, in which like reference numerals represent like components throughout the various figures.

### BRIEF DESCRIPTION OF THE FIGURES

FIG. 1 is a perspective view of an exemplary embodiment of the present invention.

FIG. 2 is a cross-sectional view of the embodiment shown in FIG. 1 configured on a hemodialysis catheter.

FIG. 3 is a cross-sectional view of the embodiment shown in FIG. 1 in place within a patient.

FIG. 4 is a schematic diagram of the general flow of a biocompatible filler into an exemplary embodiment of the expandable chamber of the present invention.

FIG. 5 is a sectional view of the embodiment shown in FIG. 1 with the expandable chamber in an unexpanded state.

FIG. 6 is a sectional view of the embodiment shown in FIG. 1 with the expandable chamber in an expanded state.

FIG. 7 is a perspective view of another exemplary embodiment of the present invention.

### DETAILED DESCRIPTION OF THE PREFERRED EMBODIMENTS

Illustrative preferred embodiments of the present invention as shown in the FIGs. comprise a stabilizing device 10 that can be configured with a catheter 30, such that the stabilizing device 10 can apply pressure to subcutaneous tissue 42 within a subcutaneous tunnel 40, thus helping to stabilize the catheter 30 in place within the subcutaneous tunnel 40. Illustrative preferred embodiments of the present invention also include catheters comprising the stabilizing device 10 and methods for employing the stabilizing device 10 to stabilize catheters, and in particular hemodialysis catheters, within the subcutaneous tunnel 40 of a patient. While the invention is described herein in conjunction with the preferred and illustrative embodiments, it will be understood that the invention is not limited to these embodiments.

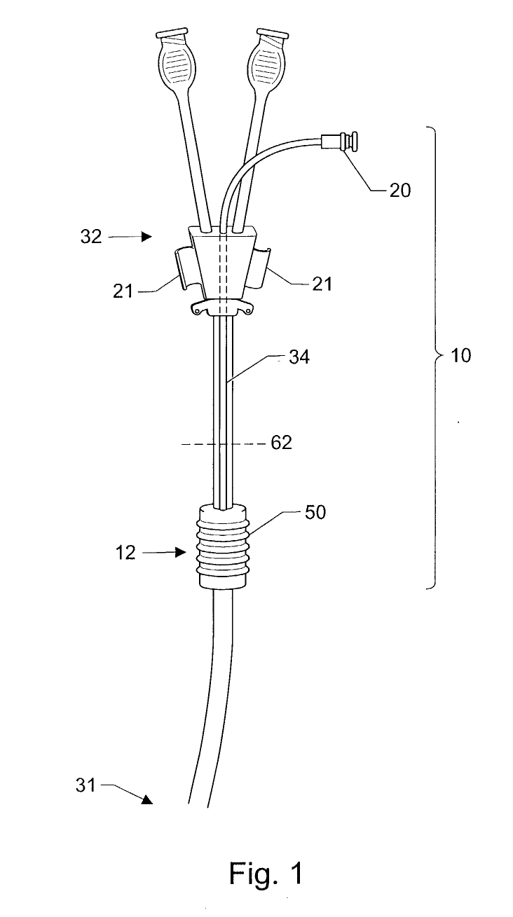

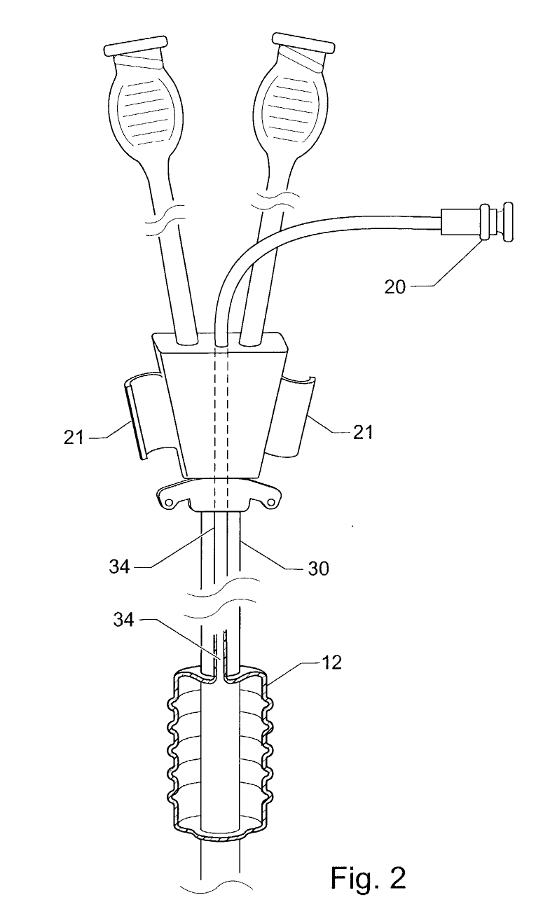

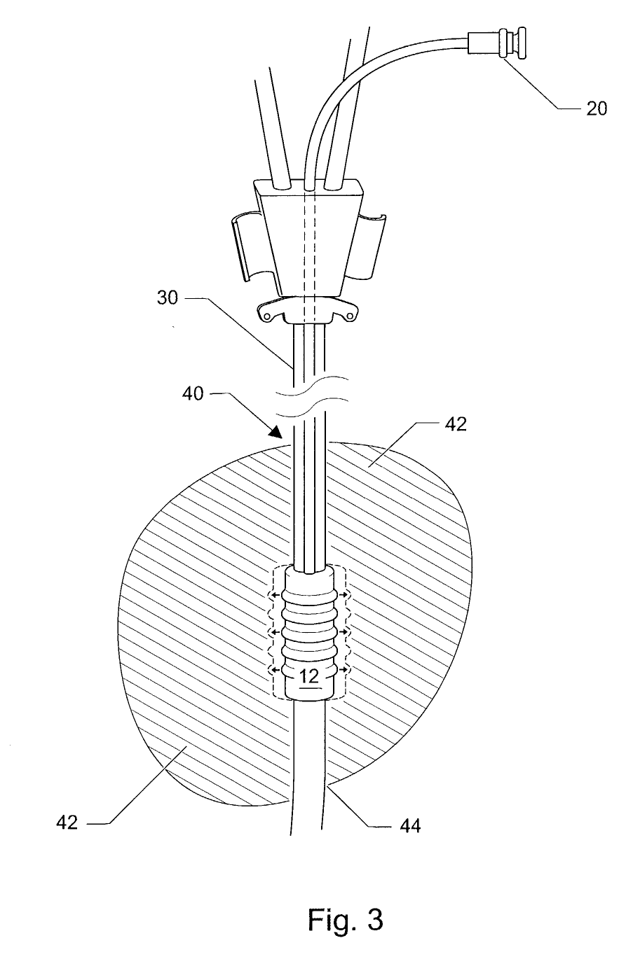

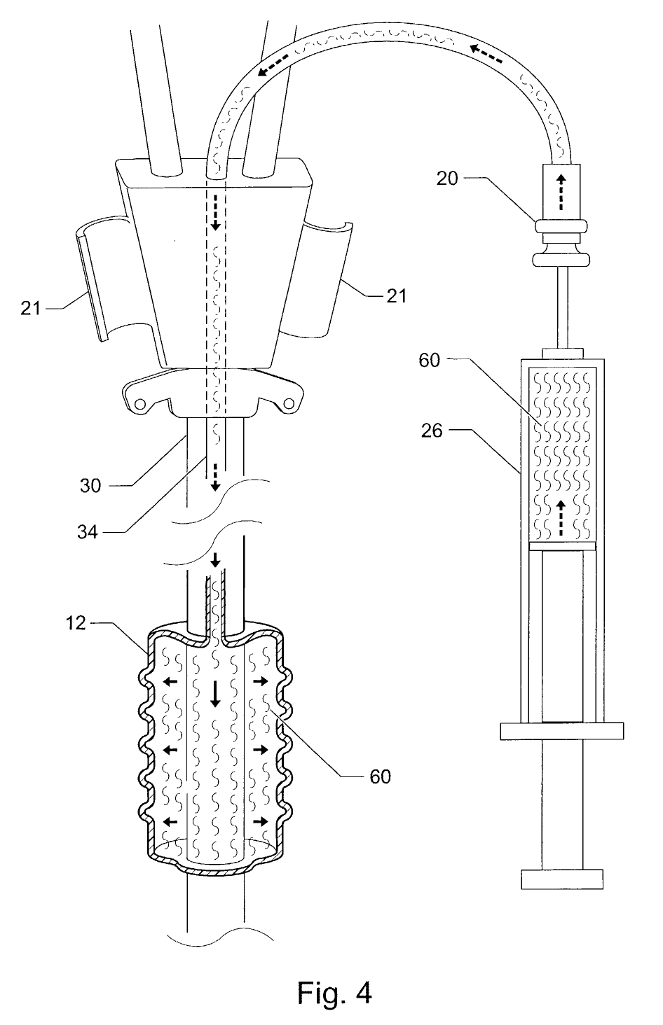

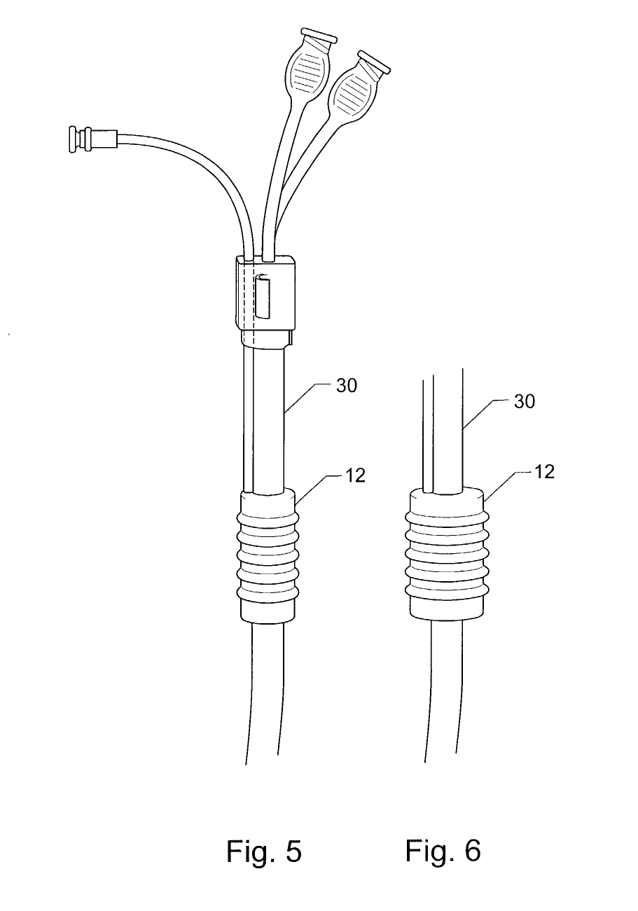

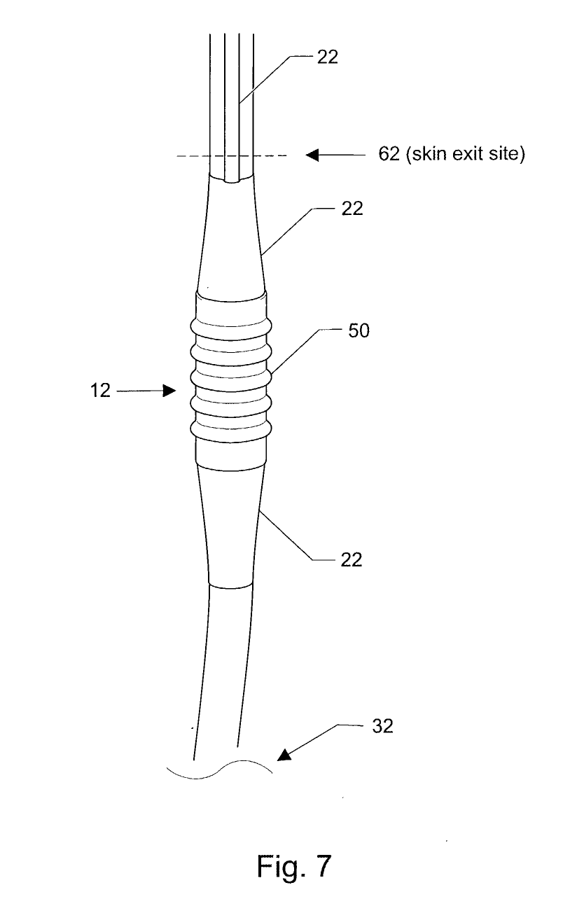

Referring now generally to FIGS. 1-6, exemplary embodiments of the present invention are shown. FIG. 1 is a perspective view of an exemplary stabilizing device 10 that generally shows the stabilizing device 10 configured with one type of hemodialysis catheter. FIG. 2 is a cross-sectional view of an exemplary stabilizing device 10 that shows that the stabilizing device 10 does not obstruct the fluid lumens(s) 34 of the catheter 30. FIG. 3 is perspective view of an exemplary stabilizing device 10 within the subcutaneous tunnel 40 of a patient and illustrates the general stabilizing mechanism of stabilizing device 10. FIG. 4 is a perspective view of an exemplary stabilizing device 10 illustrating the general flow of a biocompatible filler 60 to inflate chamber 12. FIG. 5 is a sectional view of an exemplary stabilizing device 10 with chamber 12 in an unexpanded or contracted state. FIG. 6 is a sectional view of an exemplary stabilizing device 10 with chamber 12 in an expanded or inflated state. FIG. 7 is another embodiment of the stabilizing device 10 with end pieces 22.

Although the catheter 30 as shown in the figures is of one generic type of hemodialysis catheter, it is understood that the stabilizing device 10 can be used and configured with any type of catheter. Such catheters include, but are not limited to, single lumen catheters, double lumen catheters, and multiple lumen catheters. Examples of hemodialysis catheter that can be configured with the stabilizing device 10 are shown in U.S. Pat. No. 4,134,402 to Mahurkar, U.S. Pat. No. 4,643,711 to Bates, U.S. Pat. No. 5,053,023 to Martin, U.S. Pat. No. 5,405,320 to Twardowski et al., U.S. Pat. No. 5,947,953 to Ash et al. and U.S. Pat. No. 6,001,079 to Pourchez, which are herein incorporated by reference. One of ordinary skill in the art can select a catheter 30 suitable for configuration with the present invention without undue experimentation.

FIG. 1 shows one catheter 30 with the stabilizing device 10 configured therewith. As shown, the stabilizing device 10 comprises an expandable chamber 12 with an exterior surrounding surface 14 and an inflation means 21 for inflating the 12. As used herein, the term “expandable” is meant to be a general term for the capability of being expanded, enlarged, inflated, or pumped up. Further, as used herein the term “inflate” is not used to limit the expansion of an the chamber to means such as by the infusion of a fluid, and can include the infusion of substances such as solids, liquids, or gases.

Chamber 12 can be dimensioned to surround at least a portion of catheter 30 and preferably circumferentially surrounds a portion of catheter 30 internally of the skin exit site 62 relative to the patient. Inflation means 21 for inflating chamber 12 preferably is positioned externally of the exit site relative to the patient as the inflation means 21 is employed by the practitioner to control the inflation of chamber 12. Chamber 12 and inflation means 21 can be directly attached or coupled to each other and to catheter 30. Optionally, the stabilizing device 10 can have a mechanism for securing the inflation means 21 to catheter 30.

Conventional catheters 30 typically have a proximal end 31 that remains outside of the patient's body and a distal end 32 that is inserted into the patient's body, it is convenient to describe the position of the stabilizing device 10 with respect to these two ends 31, 32. More particularly, chamber 12 is generally positioned on catheter 30 such that when the distal end 32 of catheter 30 is positioned in the subcutaneous tunnel for catheterizing a blood vessel, chamber 12 is positioned external to the catheterized part of the blood vessel, but below the skin exit site 62. Chamber 12 is proximally positioned on catheter 30 such chamber 12 is positioned within the subcutaneous tissue 42, and more preferably within the subcutaneous tunnel 40 formed during catheterization. In this embodiment, as a consequence of the positioning of chamber 12, chamber 12 will rest below the exit site 62 when catheter 30 is placed in the patient.

Preferably, the configuration of the stabilizing device 10 relative to catheter 30 does not affect the function of catheter 30. As shown in FIG. 3, chamber 12 can be configured so that it does not occlude, obstruct, or interfere with the structure of the fluid lumens(s) 34 of catheter 30. Preferably, chamber 12 does not change the diameter 36 of the fluid lumens(s) 34 of catheter 30 in either the deflated or inflated states. Specifically, as chamber 12 is positioned on the outside of the walls 38 of catheter 30, chamber 12 expands outward and does not expand inward towards or protrude into catheter 30 itself or interfere with the fluid flow within catheter 30 itself. Preferably, the means for inflating 21 chamber 12 also is outside the fluid lumens(s) 34 of catheter 30.

The stabilizing device 10 helps stabilize catheter 30 in place by applying pressure against the subcutaneous tissue 42 within the subcutaneous tunnel 40. More specifically, as shown in FIG. 3, when chamber 12 is inflated, exterior surrounding surface 14 applies pressure (indicated by arrows) to the subcutaneous tissue 42. More particularly, when chamber 12 is inflatable to a desired state, the exterior surrounding surface 14 of chamber 12 applies a level and constant pressure to the surrounding subcutaneous tissue 42. The pressure exerted from the inflated chamber 12 helps stabilize catheter 30 in place within the subcutaneous tunnel 40 by reducing the degrees of freedom and the level of space freedom available to catheter 30. In other words, the inflated stabilizing device 10 can be said to have anchored catheter 30 in place within the subcutaneous tunnel 40.

Further, additional stabilization of catheter 30 in position within subcutaneous tunnel 40 can be derived from the frictional force applied to the subcutaneous tissue 42 by the exterior surrounding surface 14 of chamber 12. For example, in one embodiment, the exterior surrounding surface 14 can have a ribbed structure as shown by reference numeral 50 on FIG. 1, or by any other type of contour. In another embodiment, the exterior surrounding surface 14 can have a fibrous covering with a rough texture. In yet another embodiment, the exterior surrounding surface 14 can comprise foam pads. In still another embodiment, the exterior surrounding surface 14 can have a smooth texture. One of ordinary skill in the art can select the exterior surrounding surface 14 for chamber 12 that will provide optimal stabilization for the configured catheter 30.

One means for inflating chamber 12 is through the use of an inflation port 30 and a syringe 26. As shown in FIG. 5, in this embodiment shown syringe 26 is employed to introduce a biocompatible filler 32 into chamber 12. More particularly, syringe 26 is inserted into an inflation port 20 and the biocompatible filler 32 is ejected from syringe 26 into chamber 12. Inflation port 20 can be part of or connected directly to chamber 12. Alternatively, inflation port 20 can have a passage 64 connecting inflation port 20 to chamber 12 through which biocompatible filler 32 can flow. Preferably, inflation port 20 has a valve (not shown) or the like that is closed when syringe 26 is not inserted therein and that is opened when syringe 26 is inserted therein to prevent biocompatible filler 32 from being ejected from or leaking out of chamber 12.

In one preferred embodiment, the practitioner also can use inflation port 20 and syringe 26 as a means to deflate or adjust the amount of inflation of chamber 12. More particularly, the practitioner can use syringe 26 to remove some or all of biocompatible filler 32 from chamber 12. Specifically, the practitioner can insert syringe 26 into inflation port 20 and remove some or all of biocompatible filler 32 from chamber 12. The deflation of chamber 12 is particularly useful in the allowing for the removal of catheter 30 from the patient.

One advantage of this type of inflation or radial expansion system is that it that allows for the controlled introduction and removal of the biocompatible filler 32 into the chamber 12 thus allowing the practitioner to control the amount of biocompatible filler 32 within chamber 12 and, hence, the amount of expansion of chamber 12. For example, FIG. 5 illustrates the stabilizing device 10, wherein chamber 12 has been inflated by the introduction of biocompatible filler 32 and is in an inflated position or state. FIG. 6 illustrates the stabilizing device 10, wherein chamber 12 is in a deflated position or state. The controlled introduction of biocompatible filler 32 through inflation port 30 allows for the controlled expansion of chamber 12 to a degree selected by the practitioner. More particularly, a practitioner can inject just enough biocompatible filler 32 so that catheter 30 is securely in place, and not so much biocompatible filler 32 such that chamber 12 expands too much and either breaks or damages subcutaneous tissue 42.

A catheter 30 comprising stabilizing device 10 can be easier to withdraw from a subcutaneous tunnel 40. After chamber 12 is deflated (which causes chamber 12 to contract), catheter 30 can be removed from the subcutaneous tunnel 40 without needless surgery by hand withdrawal or standard methods. After the subcutaneous tissue 42 has been separated from catheter 30 and the configured stabilizing device 10, the practitioner can without undue impedance remove catheter 30 from the patient and thereafter close the area. Alternatively, it may be that by the mere deflation of chamber 12, catheter 30 becomes destabilized enough to be removed from the subcutaneous tunnel 40 merely by gently pulling catheter 30 by hand.

In an alternative embodiment of the stabilizing device 10 as shown in FIG. 7, there can be end pieces 22 of a tapered shape flanking the ends of chamber 12. End pieces 22 can be useful in promoting the passage of chamber 12 through the subcutaneous tunnel 40 of the patient. More particularly, tapered ends pieces 22 can help prevent the edges of chamber 12 from snagging, dragging or catching on the subcutaneous tissue 42 within the subcutaneous tunnel 40. Preferably, end pieces 22 are generally ramp-like and taper from a position approximately flush with catheter 30 at a point distal from chamber 12 to a position approximately flush with chamber 12 when chamber 12 is in a deflated state. It is contemplated that the stabilizing device 10 can be manufactured with or without end pieces 22.

Stabilizing device 10 further can comprise or be manufactured from or with materials to prevent bacterial growth. For example, a portion of stabilizing device 10 or chamber 12 can be coated with a Decron® polymer or other similar material to impede bacterial growth. Other materials such as silver or antimicrobial substances can be placed proximally to chamber 12 or throughout the device to reduce the chance of infection in the area in which catheter 30 is placed.

In operation and use, the stabilizing device 10 is adapted to allow a practitioner to stabilize catheter 30 relative to a patient, that is relative to subcutaneous tissue 42 within subcutaneous tunnel 40. Preferably, after catheter 30 has been inserted through the patient's skin and subcutaneous tissue 42 into the appropriate blood vessel (by techniques such as the Seldinger technique) and chamber 12 has been appropriately placed for optimal position, the practitioner begins to inflate chamber 12 by injecting biocompatible filler 32 into chamber 32. This can be done by connecting a syringe 26 to the inflation port 20 and ejecting an amount of biocompatible filler 32 from the syringe 26 into chamber 12. As chamber 12 fills with biocompatible filler 32 and is inflated, chamber 12 eventually begins to apply pressure to the patient's subcutaneous tissue 42. This pressure helps secure chamber 12, and hence catheter 30, to the patient's subcutaneous tissue 42. When the practitioner needs to remove catheter 30 from the patient, the practitioner can remove catheter 30 by removing biocompatible filler 32 from, and thus deflating, chamber 12, which reduces the pressure on the subcutaneous tissue 42, allowing catheter 30 to become destabilized and removed as necessary and as know by those with skill in the art.

Although the description of the inflation of chamber 12 is disclosed herein as generally radially, chamber 12 can be inflated in any manner and direction that will secure catheter 30 within the subcutaneous tissue 42. For example, chamber 12 also can be constructed to expand lengthwise, radially and lengthwise, or for only a portion of chamber 12 to expand radially and/or lengthwise, or in any other direction that will secure catheter 30 within the subcutaneous tissues 42.

Methods for manufacturing the stabilizing device 10 now are evident to those with ordinary skill in the art. In one illustrative method, catheter 30 can be manufactured independently of the stabilizing device 10 and subsequently the stabilizing device 10 can be affixed to catheter 30. For example, the stabilizing device 10 can be manufactured using an ordinary molding method and later affixed to catheter 30 by means of an adhesive, ultrasonic welding, or other type of adhesive method obvious to those with skill in the art. In another illustrative method, catheter 30 and the stabilizing device 10 can be manufactured as a single manufactured unit. For example, chamber 12 with exterior surrounding surface 14, passage 64 if included, and inflation port 14 all can be co-manufactured with catheter 30.

The stabilizing device 10 can be formed of a biocompatible material, such as polyethylene or any standard material known to those skilled in the art. Preferred biocompatible materials include low pressure, relatively soft or flexible polymeric materials. Other materials such relatively rigid stiff high pressure polymeric materials also can be used in certain circumstances. Suitable materials include but are not limited to thermoplastic polymers, thermoplastic elastomers, polyethylene, various co-polymers and blends of polyethylene, ionomers, polyesters, polyurethanes, polycarbonates, polyamides, poly-vinyl chloride, acrylonitrile-butadiene-styrene copolymers, polyether-polyester copolymers, polyetherpolyamide copolymers, thermoset polymeric materials, poly(ethylene terephthalate), polyimide, thermoplastic polyimide, polyphenylene sulfides, polypropylene and rigid polyurethanes, or combinations thereof.

It is understood that material used for the manufacture of chamber 12 can include plastics and biomaterials acceptable to those with ordinary skill in the art. The materials preferably should be selected from materials that are flexible and resilient and compatible with the human skin and tissues (that is, non-toxic and non-irritating). Preferably, the material selected can have a very low permeability and a high degree of elasticity such that it will inflated when a biocompatible filler 32 is introduced and it will contract when the biochemical filler 32 is removed.

Chamber 12 can have an array of shapes and dimensions. For example, chamber 12 can have cylindrical shape, a circular shape, a disk shape or be shaped like a torus. Preferably, a cylindrical shape is used with most catheters 30. The size of chamber 12 can be dependant on the specific catheter 30 to which the stabilizing device 10 and chamber 12 are configured. One of ordinary skill in the art can select a size and shape for both the stabilizing device 10 and chamber 12 with undue experimentation.

It is understood that chamber 12 can be filled with any biocompatible filler 32 known to those with ordinary skill in the art. As the leakage of air or other gases into a patient, particularly into a patient's heart, can have serious consequences, biocompatible filler 32 preferably is not air or other gases. One preferable biocompatible filler 32 is a sterile saline solution. Other biocompatible fillers can include hydrogels and other biocompatible liquids. Biocompatible filler 32 preferably is non-toxic and non-irritating to human skin and tissues.

Although embodiments of the present invention are generally disclosed in the context of hemodialysis catheters, it is understood that such embodiments can be applied to other catheters that are used for procedures that require that fluid, blood, medicated solution, or other solutions be removed and introduced into a patient. Such procedures include, but are not limited to, hemodialysis, perfusion, chemotherapy, and plasmapheresis.

The above detailed description of the preferred embodiments, examples, and the appended figures are for illustrative purposes only and are not intended to limit the scope and spirit of the invention, and its equivalents, as defined by the appended claims. One skilled in the art will recognize that many variations can be made to the invention disclosed in this specification without departing from the scope and spirit of the invention.

What is claimed is:

1. A device for stabilizing a catheter relative to a patient's tissue comprising:
a. an expandable chamber having a surrounding surface; and
b. a means for expanding the expandable chamber,
wherein the chamber is attached to the catheter at a position that will be inside of the patient's body when the catheter is in a medically appropriate working position, the expandable chamber is in fluid connection with the means for expanding the expandable chamber, and the expandable chamber is reversibly expandable from a contracted state to an expanded state, whereby the expansion of the expandable chamber from the contracted state to the expanded state causes the application of pressure to the patient's tissue for stabilizing the catheter relative to the patient's tissue.

2. The device as claimed in claim 1, wherein the medically appropriate working position is within subcutaneous tissue.

3. The device as claimed in claim 2, wherein the subcutaneous tissue is within a subcutaneous tunnel.

4. The device as claimed in claim 3, wherein the subcutaneous tunnel is between a patient's skin surface and a blood vessel to be catheterized.

5. The device as claimed in claim 4, wherein the expandable chamber is not placed within the blood vessel of the patient.

6. The device as claimed in claim 5, wherein the catheter is a hemodialysis catheter.

7. The device as claimed in claim 1, wherein at least a portion of the expandable chamber is coated with infection resistance material.

8. The device as claimed in claim 1, wherein the means for expanding the expandable chamber comprises a filler and an inflation port for allowing the introduction of the filler into the expandable chamber.

9. The device as claimed in claim 8, wherein the expandable chamber expands with the introduction of the filler.

10. The device as claimed in claim 9, wherein the filler is a biocompatible material.

11. The device as claimed in claim 10, wherein the filler is selected from the group consisting of saline solutions and hydrogels.

12. The device as claimed in claim 11, wherein the filler is a saline solution.

13. The device as claimed in claim 1, wherein the expandable chamber is configured to fit around at least a portion of the catheter.

14. A device for stabilizing a hemodialysis catheter within a subcutaneous tunnel of a patient, comprising:
a. an expandable chamber having a surrounding surface, the expandable chamber being configured for positioning around the hemodialysis catheter, and the expandable chamber having a variable expansion state;
b. a means for expanding the expandable chamber upon the introduction of a biocompatible filler thereinto, the means for expanding the expandable chamber comprising an inflation port in fluid communication with the expandable chamber,
wherein the biocompatible filler is introduced into the inflation port and consequently into the expandable chamber, the expandable chamber expands from a contracted state to an expanded state;
whereby the expansion of the expandable chamber applies pressure to tissue within the subcutaneous tunnel and the pressure applied by the expandable chamber to the tissue improves the stability of the hemodialysis catheter in position within the subcutaneous tunnel.

15. The device as claimed in claim 14, wherein the subcutaneous tunnel is between a patient's skin surface and a blood vessel to be catheterized.

16. The device as claimed in claim 15, wherein the expandable chamber is not placed within the blood vessel of the patient.

17. The device as claimed in claim 14, wherein the catheter is a hemodialysis catheter.

18. The device as claimed in claim 14, wherein at least a portion of the expandable chamber is coated with infection resistance material.

19. The device as claimed in claim 14, wherein the biocompatible filler is selected from the group consisting of saline solutions and hydrogels.

20. The device as claimed in claim 14, wherein the biocompatible filler is a saline solution.

21. The device as claimed in claim 14, wherein the expandable chamber is configured to fit around at least a portion of the catheter.

22. A method for stabilizing a hemodialysis catheter to subcutaneous tissue within a subcutaneous tunnel of a patient comprising the steps of:
a. positioning a hemodialysis catheter that comprises an expandable chamber and an inflation port in fluid communication with the expandable chamber into a desired position with the patient;
b. inflating the expandable chamber to a desired inflation level by introducing a biocompatible filler into the expandable chamber through the inflation port;
whereby inflating the expandable chamber to the desired inflation level improves the stability of the catheter in place with respect to the subcutaneous tissue within subcutaneous tunnel of the patient.

23. The method as claimed in claim 22, wherein the expandable chamber is inflated to a preset inflation level.

24. The method as claimed in claim 23, wherein the catheter is positioned in the patient using the Seldinger procedure.

25. A method for manufacturing a hemodialysis catheter unit comprising a means for securing the catheter relative to a patient's body, comprising the steps of:
a. providing a catheter that removes and introduces a fluid into a patient;
b. constructing a stabilizing device comprising an expandable chamber, an inflation port, and a means for fluidly connecting the expandable chamber and the inflation port; and
c. affixing the stabilizing device to the catheter at a position on the catheter such that the stabilizing device will rest proximal to the subcutaneous tissue of the patient when the catheter is placed in a medically acceptable working position in the patient.

26. The device as claimed in claim 25, wherein at least a portion of the expandable chamber is coated with infection resistance material.

27. A catheter that removes and introduces fluids into the patient, the catheter comprising a stabilizing device, wherein the stabilizing device comprises an expandable chamber and an inflation means in fluid communication with the expandable chamber and wherein the inflation means is configured to allow the introduction of a biocompatible filler through the inflation port into the expandable chamber, and wherein the expandable chamber inflates as the biocompatible fluid is introduced into the expandable chamber.

28. The catheter as claimed in claim 26, wherein the catheter is a hemodialysis catheter.
